(19) United States
(12) Patent Application Publication
(10) Pub. No.: US 2025/0256101 A1
(43) Pub. Date: Aug. 14, 2025

(54) ELECTRIC-FIELD DIRECTED NERVE REGENERATION

(71) Applicant: UNIVERSITY OF SOUTHERN CALIFORNIA, Los Angeles, CA (US)

(72) Inventors: Gianluca LAZZI, Los Angeles, CA (US); Kimberly GOKOFFSKI, Los Angeles, CA (US); Pooyan PAHLAVAN, Los Angeles, CA (US)

(73) Assignee: UNIVERSITY OF SOUTHERN CALIFORNIA, Los Angeles, CA (US)

(21) Appl. No.: 19/052,850

(22) Filed: Feb. 13, 2025

Related U.S. Application Data
(60) Provisional application No. 63/553,601, filed on Feb. 14, 2024.

(51) Int. Cl. A61N 1/36

(52) U.S. Cl. CPC A61N 1/36046; A61N 1/36042

(57) ABSTRACT

A retinal ganglion cell (RGC) stimulation system for an optic nerve. The system can comprise a ground electrode, a stimulation electrode, a voltage or current source connected to both the ground electrode and the stimulation electrode and configured to stimulate the stimulation electrode with an electrical waveform having a first voltage and a first current, and a controller connected to the voltage or current source and controlling the first voltage and the first current of the electrical waveform.

### CROSS-REFERENCE TO RELATED APPLICATIONS

This application claims the benefit of and priority to U.S. Provisional Application No. 63/553,601, filed on Feb. 14, 2024, which is herein incorporated by this reference in its entirety.

### STATEMENT AS TO FEDERALLY SPONSORED RESEARCH

This invention was made with government support under grants EY031797 and EY035375 from the National Institutes of Health (NIH), W81XWH-22-1-0743 from the Department of Defense (DOD) and 2121164 from the National Science Foundation (NSF). The government has certain rights in this invention.

### BACKGROUND INFORMATION

Significant interest exists in harnessing applied electrical fields into a therapy that can direct cellular processes such as nerve regeneration and wound healing. Although electric field stimulation with Direct Currents (DC) has shown promise to regenerate nerves, this cannot be employed in therapy since DC can introduce net charge into the tissue, thereby leading to tissue damage.

Therefore, in view of the above, there is a need for new electric field based therapies for cell regeneration.

### SUMMARY

Exemplary embodiments of the present disclosure include a retinal ganglion cell (RGC) stimulation system for an optic nerve, the system comprising: a ground electrode located on a first side of optic nerve damage; a stimulation electrode located on a second side of the optic nerve damage; a voltage or current source connected to both the ground electrode and the stimulation electrode and configured to stimulate the stimulation electrode with an electrical waveform having a first voltage and a first current; and a controller connected to the voltage or current source and controlling the first voltage and the first current of the electrical waveform.

In certain embodiments the first side of the optic nerve damage comprises an area behind an eye and the second side of the optic nerve damage comprises an area proximate to a frontal lobe of a brain or a temporal lobe of the brain. In particular embodiments the first side of the optic nerve damage comprises an area in front of an eye and the second side of the optic nerve damage comprises an area proximate to an occipital lobe of a brain. In some embodiments the first side of the optic nerve damage comprises an area in front of an eye and the second side of the optic nerve damage comprises an area proximate to or within a nasal cavity. In specific embodiments the ground electrode comprises a contact lens and the area in front of the eye comprises a cornea of the eye.

Certain embodiments further comprise a molecular scaffold located at the optic nerve damage. In particular embodiments the molecular scaffold excludes fibroblasts from the optic nerve damage. Some embodiments further comprise a Ciliary neurotrophic factor (CTNF) containing implant placed proximate to the optic nerve damage. In specific embodiments the CTNF containing implant promotes survival of donor RGC cells.

In certain embodiments a distribution of RGC subtypes in the optic nerve is different than a distribution of RGC subtypes in the donor RGC cells. In particular embodiments the electrical waveform is an asymmetric charge balanced biphasic waveform configured to promote neuronal regeneration of a retinal ganglion cell axon, wherein the first voltage changes over time. In some embodiments the RGC stimulation system promotes neuronal regeneration of a retinal ganglion cell axon in the optic nerve. In specific embodiments the RGC stimulation system promotes cell health in the optic nerve.

In certain embodiments the ground electrode is connected to an optical nerve at an optic tract, and wherein the stimulation electrode is connected to the optical nerve or corneal surface, wherein a voltage gradient is induced along the optic nerve to direct neuronal regeneration of a retinal ganglion cell axon between the stimulation electrode and the ground electrode. In particular embodiments the ground electrode is connected to an optical nerve within the nasal sinus, and wherein the stimulation electrode is connected to the optical nerve or corneal surface, wherein a voltage gradient is induced along the optic nerve to direct neuronal regeneration of a retinal ganglion cell axon between the stimulation electrode and the ground electrode.

In some embodiments the ground electrode is connected to the scalp near the occipital lobe, and wherein the stimulation electrode is connected to the optical nerve or corneal surface, wherein a voltage gradient is induced along the optic nerve to direct neuronal regeneration of a retinal ganglion cell axon between the stimulation electrode and the ground electrode. In specific embodiments the ground electrode and the stimulation electrode are selected from a group consisting of: (i) the ground electrode and the stimulation electrode are both platinum, (ii) the ground electrode and the stimulation electrode are both tungsten, and (iii) the ground electrode is tungsten and the stimulation electrode is platinum. In certain embodiments the electrical waveform comprises both positive pulses and negative pulses relative to a ground potential of the ground electrode, wherein the positive pulses have greater amplitude and shorter duration and the negative pulses have lower amplitude and longer duration.

In particular embodiments the electrical waveform comprises both positive pulses and negative pulses relative to a ground potential of the ground electrode, wherein the negative pulses have greater amplitude and shorter duration and the positive pulses have lower amplitude and longer duration. In some embodiments the electrical waveform comprises both positive pulses and negative pulses relative to a ground potential of the ground electrode, wherein the positive pulses stimulate neuronal regeneration of a retinal ganglion cell axon and the negative pulses restore a charge balance. In specific embodiments the electrical waveform comprises both positive pulses and negative pulses relative to a ground potential of the ground electrode, wherein the positive pulses and the negative pulses are of a same pulse length in time and a same pulse amplitude, wherein a combination of the positive pulses and the negative pulses promotes cellular health of a cell of the optic nerve.

In certain embodiments the electrical waveform stimulates RGC axon growth toward an electrode of the ground electrode and the stimulation electrode has a positive voltage relative to the ground electrode. In particular embodiments the electrical waveform is an asymmetric cathodic-first charge balanced biphasic waveform.

Exemplary embodiments include a method of retinal ganglion cell (RGC) stimulation for an optic nerve comprising: providing a ground electrode; providing a stimulation electrode; providing a voltage or current source connected to both the ground electrode and the stimulation electrode and configured to stimulate the stimulation electrode with an electrical waveform having a first voltage and a first current; and controlling by a controller connected to the voltage or current source, the first voltage and the first current of the electrical waveform to generate a waveform, wherein the first voltage changes over time.

In certain embodiments generating the waveform comprises: generating both positive pulses and negative pulses relative to a ground potential of the ground electrode; stimulating, by the positive pulses, neuronal regeneration of a retinal ganglion cell axon; and restoring, by the negative pulses, a charge balance. In particular embodiments the negative pulses have greater amplitude and shorter duration and the positive pulses have lower amplitude and longer duration. In some embodiments the waveform is an asymmetric charge balanced biphasic waveform to promote neuronal regeneration of a retinal ganglion cell axon in the optic nerve. In specific embodiments the waveform is a symmetric charge balanced biphasic waveform to promote cell health in the optic nerve.

In certain embodiments providing the voltage or current source further comprises providing an active circuit and the method of RGC stimulation further comprises: increasing, by the active circuit, the first voltage between the ground electrode and the stimulation electrode; and limiting, by the active circuit, the first current between the ground electrode and the stimulation electrode.

Exemplary embodiments include a system for electric-field directed nerve stimulation comprising: a first electrode; a second electrode; a voltage or current source connected to both the first electrode and the second electrode and configured to stimulate the first electrode with an electrical waveform having a first voltage and a first current; and a controller connected to the voltage or current source and controlling the first voltage and the first current of the electrical waveform to induce a voltage differential across a nerve for regeneration, wherein the electrical waveform comprises at least one of an asymmetric cathodic-first charge balanced biphasic waveform.

Exemplary embodiments include a system for electric-field directed nerve stimulation comprising: a first electrode; a second electrode; a voltage or current source connected to both the first electrode and the second electrode and configured to stimulate the first electrode with an electrical waveform having a first voltage and a first current; and a controller connected to the voltage or current source and controlling the first voltage and the first current of the electrical waveform to induce a voltage differential across a nerve to promote cellular health, wherein the electrical waveform comprises a symmetric charge balanced biphasic waveform.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover anon-exclusive inclusion, such that a process, method, system, article, device, or apparatus that comprises a list of elements is not necessarily limited to those elements but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

The terms “couple,” “coupled,” “couples,” “coupling,” and the like should be broadly understood and refer to connecting two or more elements mechanically and/or otherwise. Two or more electrical elements may be electrically coupled together, but not be mechanically or otherwise coupled together. Coupling may be for any length of time, e.g., permanent or semi-permanent or only for an instant. “Electrical coupling” and the like should be broadly understood and include electrical coupling of all types. The absence of the word “removably,” “removable,” and the like near the word “coupled,” and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “real-time” can, in some embodiments, be defined with respect to operations carried out as soon as practically possible upon occurrence of a triggering event. A triggering event can include receipt of data necessary to execute a task or to otherwise process information. Because of delays inherent in transmission and/or in computing speeds, the term “real time” encompasses operations that occur in “near” real time or somewhat delayed from a triggering event. In a number of embodiments, “real time” can mean real time less a time delay for processing (e.g., determining) and/or transmitting data. The particular time delay can vary depending on the type and/or amount of the data, the processing speeds of the hardware, the transmission capability of the communication hardware, the transmission distance, etc. However, in some embodiments, the time delay can be less than approximately one second, two seconds, five seconds, or ten seconds.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein (and in particular, the inclusion of any particular embodiment, feature or function within the Abstract or Summary is not intended to limit the scope of the invention to such embodiment, feature or function). Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention. The disclosures of all patents, patent applications and publications cited herein are hereby incorporated herein by reference in their entireties, to the extent that they are consistent with the present disclosure set forth herein.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

At least a portion of embodiments discussed herein can be implemented using a computer communicatively coupled to a network (for example, the Internet), another computer, or in a standalone computer. As is known to those skilled in the art, a suitable computer can include a processor or central processing unit (“CPU”), at least one read-only memory (“ROM”), at least one random access memory (“RAM”), at least one hard drive (“HD”), and one or more input/output (“I/O”) device(s). The I/O devices can include a keyboard, monitor, printer, electronic pointing device (for example, mouse, trackball, stylist, touch pad, etc.), or the like.

ROM, RAM, and HD are computer memories for storing computer-executable instructions executable by the CPU or capable of being complied or interpreted to be executable by the CPU. Suitable computer-executable instructions may reside on a computer readable medium (e.g., ROM, RAM, and/or HD), hardware circuitry or the like, or any combination thereof. Within this disclosure, the term “computer readable medium” or is not limited to ROM, RAM, and HD and can include any type of data storage medium that can be read by a processor. For example, a computer-readable medium may refer to a data cartridge, a data backup magnetic tape, a floppy diskette, a flash memory module or drive, an optical data storage drive, a CD-ROM, ROM, RAM, HD, or the like. Software implementing some embodiments disclosed herein can include computer-executable instructions that may reside on a non-transitory computer readable medium (for example, a disk, CD-ROM, a memory, etc.). Alternatively, the computer-executable instructions may be stored as software code components on a direct access storage device array, magnetic tape, floppy diskette, optical storage device, or other appropriate computer-readable medium or storage device.

Any suitable programming language can be used to implement the routines, methods or programs of embodiments of the invention described herein, including the custom script. Other software/hardware/network architectures may be used. For example, the software tools and the custom script may be implemented on one computer or shared/distributed among two or more computers in or across a network. Communications between computers implementing embodiments can be accomplished using any electronic, optical, radio frequency signals, or other suitable methods and tools of communication in compliance with known network protocols. Additionally, any signal arrows in the drawings/figures should be considered only as exemplary, and not limiting, unless otherwise specifically noted. Based on the disclosure and teachings provided herein, a person of ordinary skill in the art will appreciate other ways and/or methods to implement the invention.

Any embodiment of any of the present methods, composition, kit, and systems may consist of or consist essentially of—rather than comprise/include/contain/have—the described steps and/or features. Thus, in any of the claims, the term “consisting of” or “consisting essentially of” may be substituted for any of the open-ended linking verbs recited above, in order to change the scope of a given claim from what it would otherwise be using the open-ended linking verb.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” or “approximately” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, including the claims that follow, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein, the meaning of “in” includes “in” and “on” unless the context clearly dictates otherwise.

As used herein, “patient” or “subject” includes mammalian organisms, such as human and non-human mammals, for example, but not limited to, rodents, mice, rats, non-human primates, companion animals such as dogs and cats as well as livestock, e.g., sheep, cow, horse, etc. Therefore, for example, although the described embodiments illustrate use of the present methods on humans, those of skill in the art would readily recognize that these methods and compositions could also be applied to veterinary medicine as well as on other animals.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. The patent or application file may contain at least one drawing executed in color. Copies of this patent or patent application publication with color drawing(s) will be provided by the Office upon request and payment of the necessary fee.

FIG. 1 illustrates an exemplary system according to an embodiment;

FIG. 2 illustrates an exemplary waveform implemented in an embodiment;

FIG. 3 illustrates an exemplary heatmap summarizing data from exemplary embodiments;

FIG. 4 illustrates a graphical representation of nerve growth induced by different exemplary waveforms;

FIG. 5 illustrates a graphical representation of nerve growth induced by different exemplary waveforms; and

FIG. 6 illustrates results of exemplary treatment with systems described herein.

FIG. 7 illustrates stimulation and ground electrodes used for EF guided optic nerve regeneration.

FIG. 8 illustrates the Mida heterogenous computational head model utilized in the simulations.

FIG. 9 illustrates the location of measuring electrodes, stimulation electrodes and ground electrodes in cadaveric experiments to measure induced voltage along the optic nerve.

FIG. 10 illustrates a graph showing asymmetric, biphasic, input current in cadaveric ex-vivo induced voltage measurement.

FIG. 11 illustrates computed voltage (mV) distribution at the orbitomeatal plane.

FIG. 12 illustrates a graph showing simulated voltage along the ON of a human model for different combinations of direct and indirect stimulation and ground electrodes.

FIG. 13 illustrates a graph showing simulated electric field along the ON of a human model for different combinations of direct and indirect stimulation and ground electrodes.

FIG. 14 illustrates graphs showing measured voltage along the optic nerve of human cadavers.

FIG. 15 illustrates graphs showing measured electric field along the optic nerve (curve-fitted, from experimental measurements) compared against simulated electric field in a heterogeneous human head model.

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

### DETAILED DESCRIPTION OF THE INVENTION

A number of embodiments can include a retinal ganglion cell (RGC) stimulation system for an optic nerve. The system can comprise a ground electrode; a stimulation electrode; a voltage or current source connected to both the ground electrode and the stimulation electrode and configured to stimulate the stimulation electrode with an electrical waveform having a first voltage and a first current; and a controller connected to the voltage or current source and controlling the first voltage and the first current of the electrical waveform.

Some embodiments can include a method of retinal ganglion cell (RGC) stimulation for an optic nerve. The method can comprise providing a ground electrode; providing a stimulation electrode; providing a voltage or current source connected to both the ground electrode and the stimulation electrode and configured to stimulate the stimulation electrode with an electrical waveform having a first voltage and a first current; and controlling by a controller connected to the voltage or current source, the first voltage and the first current of the waveform to generate a waveform, wherein the first voltage changes over time.

Various embodiments can include a system for electric-field directed nerve stimulation. The system can comprise a first electrode; a second electrode; a voltage or current source connected to both the first electrode and the second electrode and configured to stimulate the first electrode with an electrical waveform having a first voltage and a first current; and a controller connected to the voltage or current source and controlling the first voltage and the first current of the electrical waveform to induce a voltage differential across a nerve for regeneration, wherein the electrical waveform can comprise at least one of an asymmetric cathodic-first charge balanced biphasic waveform.

Further embodiments can include a system for electric-field directed nerve stimulation. The system can comprise a first electrode; a second electrode; a voltage or current source connected to both the first electrode and the second electrode and configured to stimulate the first electrode with an electrical waveform having a first voltage and a first current; and a controller connected to the voltage or current source and controlling the first voltage and the first current of the electrical waveform to induce a voltage differential across a nerve to promote cellular health, wherein the electrical waveform comprises a symmetric charge balanced biphasic waveform.

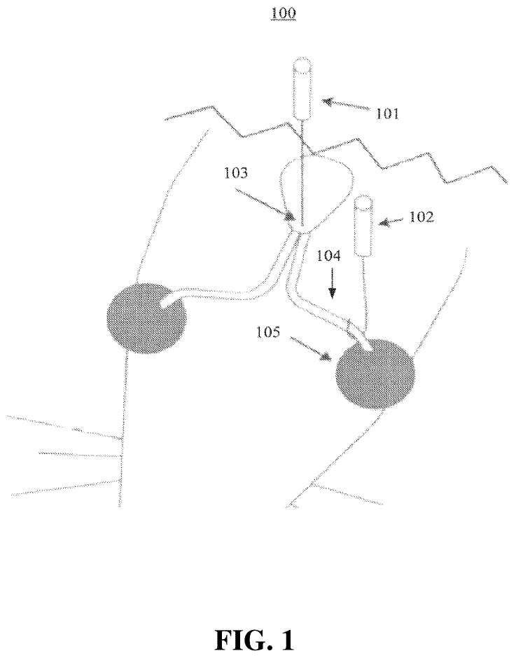

Turning to the drawings, FIG. 1 illustrates an exemplary embodiment of a system 100 for nerve regeneration. System 100 is merely exemplary and is not limited to the embodiments presented herein. System 100 can be employed in some different embodiments or examples not specifically depicted or described herein. In some embodiments, the elements of system 100 can be coupled in the arrangement presented. In other embodiments, the elements of system 100 can be coupled in any suitable arrangement. In still other embodiments, one or more of the elements of system 100 can be combined or omitted. In some embodiments, system 100 can comprise a ground electrode 101, a stimulation electrode 102, and/or a voltage or current source (not shown). Ground electrode 101 and a stimulation electrode 102 can be made from a variety of electrically conductive materials suitable for use on tissue. For example, ground electrode 101 and/or stimulation electrode 102 can be made from platinum and/or tungsten. In various embodiments, ground electrode 101 and stimulation electrode 102 can be made out of the same and/or different materials. In various embodiments, a platinum electrode can produce larger amplitudes along a nerve than a tungsten electrode.

In various embodiments, stimulation electrode can be wrapped around and/or inserted into a nerve (e.g., an optic nerve 104), thereby facilitating electrical coupling with nerve cells in the nerve. In various embodiments, stimulation electrode 102 can be placed on a first side of a nerve injury. For example, if optic nerve 104 is damaged, stimulating electrode 102 can be place behind an eye 105 of a patient. In some embodiments, stimulation electrode 102 can be placed near an axon terminal of a damaged nerve. In some embodiments, ground electrode 101 can be placed on a second side of a nerve injury and/or along a nerve tract. For example, if optic nerve 104 is damaged, ground electrode 101 can be placed along an optic nerve tract (e.g., at optic chasm 103). In some embodiments, ground electrode can be placed near a target area towards which nerve growth is desired. In various embodiments, ground electrode 101 and stimulation electrode 102 can be inserted into a patient in a stereotactic surgery.

In some embodiments, a voltage or current source can be electrically coupled to one or more of ground electrode 101 and a stimulation electrode 102. Generally speaking, a voltage or current source can be configured to produce a voltage gradient along a nerve. In various embodiments, a voltage gradient can be produced by electrifying (e.g., inducing a current into) stimulation electrode 102. In some embodiments, a voltage gradient can be used to direct neuronal (e.g., optic nerve 104) regeneration after an injury. For example, growth of retinal ganglion cells (RGCs) in an optic nerve can be directed toward ground electrode 101. In some embodiments, a voltage gradient can run from stimulation electrode 102 to ground electrode 101.

In various embodiments, system 100 can generate pulses characterized as working pulses which generate axonal regeneration, and recharging pulses which rebalance a charge in tissue. In various instances, a working pulse has a relatively lower amplitude and longer pulse width, while a recharging pulse has a relatively shorter pulse width and higher amplitude. As such, over time, the duration of tissue exposure to working pulses is greater than the duration of tissue exposure to recharging pulses, thereby causing a net axon growth associated with the working pulse to exceed the net axon growth associated with the recharging pulse. In this way, a sum of growth does not cancel. At the same time, because the recharging pulse has a higher amplitude, the net charge delivered across the tissue is null, as the recharging pulses balance the working pulses, ameliorating tissue damage.

In various embodiments, system 100 can generate positive and negative pulses with similar (e.g., same) length. In various instances, system 100 can generate positive and negative pulses with similar (e.g., same) amplitude. Thus, one may say that the system generates symmetric pulses. Rather than inducing net axon growth, such a system may promote cellular health in a cell of tissue exposed to the pulses. Such a system may encourage retinal ganglioncell survival. In further instances, the system may switch between producing asymmetric pulses and symmetric pulses. Thus, the system may alternately promote axon growth and also promote cellular health as directed by a controller. The controller may cause the system to produce different pulses of different durations and amplitudes responsive to an operator, sensors, and/or a preset program.

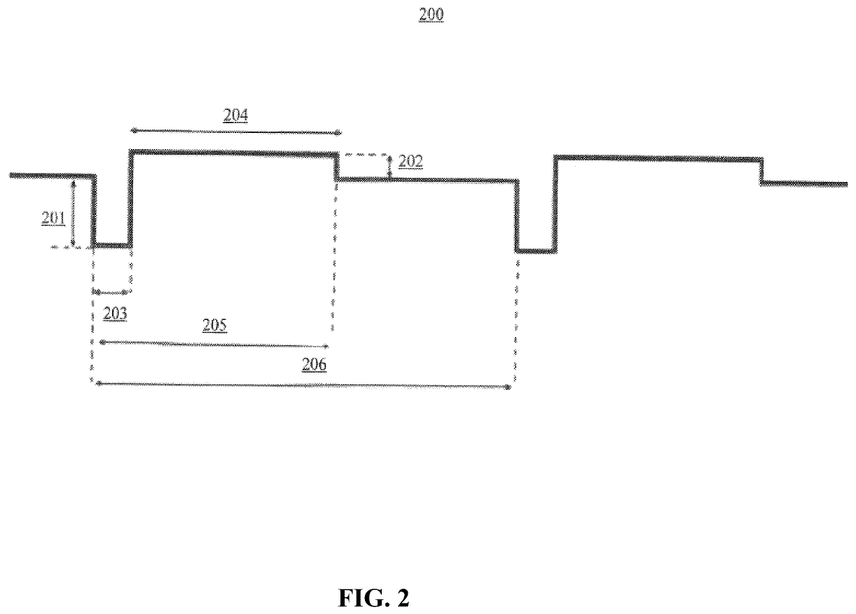

In some embodiments, a voltage or current source can be configured to generate a number of waveforms and/or electric fields configured to regenerate nerves. Turning now to FIG. 2, an exemplary waveform 200 is shown. Waveform 200 can be described by a cathodic voltage amplitude (Ve) 201, anodic voltage amplitude (VA) 202, cathodic pulse width (PWc) 203, anodic pulse width (PWA) 204, overall width (Wo) 205, period 206, and phase duration ratio (PDR). In some embodiments, properties of waveform 200 can be calculated using the equations below:

$${V}_{A} = \frac{{V}_{C}}{PDR}$$

$$PDR = \frac{{PW}_{A}}{{PW}_{C}}$$

In an exemplary embodiment, a cathodic voltage amplitude can be set to 4 V with a pulse width of 0.1 ms and an anodic voltage amplitude was set to 1 V with a pulse width of 0.4 ms. In this exemplary embodiment, a PDR of 4 can be chosen due to the existence of a threshold electric field of 100 mV/mm for eliciting cathode directed growth in RGCs.

In some embodiments, a waveform 200 can be programmed and delivered by a controller (e.g., a computer system). For example, an Agilent 33220A Arbitrary Waveform Generator (AWG) and/or a STG 4008 Multi Channel Systems created by Harvard Bioscience, Inc. can be used to generate waveform 200. In various embodiments, a Keysight DSOX2014A oscilloscope can be used to record waveforms along a nerve while in a high-resolution acquisition mode. In order to record current passing through a nerve, a resistor (e.g., 10 Q) can be added in series to targeted tissue and a voltage across it can be read.

Returning now to FIG. 2, in some embodiments, waveform 200 can comprise an asymmetric charge balanced (ACB) waveform. In some embodiments, waveform 200 can comprise a biphasic voltage waveform where voltage is changed over time. In other words, waveform 200 can comprise both positive (i.e., anodic) pulses and negative (i.e., cathodic) pulses relative to a ground potential of ground electrode 101 (FIG. 1). In some embodiments, waveform 200 can comprise a cathodic first waveform or an anodic first waveform. Pulses can be structured in a number of ways in waveform 200. In some embodiments, positive pulses have can have a greater amplitude and a shorter duration than negative pulses. Negative pulses can have a greater amplitude and a shorter duration that positive pulses. In further embodiments, positive pulses and negative pulses can have a same pulse length in time and a same pulse amplitude. Positive and negative pulses in waveform 200 can have various functions. For example, positive pulses can stimulate nerve regeneration and negative pulses restore a charge balance across the nerve and/or individual nerve cells. In this way, nerve growth can be stimulated while preventing a damaging buildup of charge in the nerve or its cells.

In some embodiments, waveform 200 can be configured to induce a calcium influx in a stimulated nerve cell. Calcium influx in a nerve cell can induce cytoskeleton (e.g., actin) polymerization. In some embodiments, waveform 200 can induce asymmetric localization and/or activation of cell surface receptors and/or channels (e.g., voltage gated Ca2+ channels). In some embodiments, calcium influx can be higher in cathode oriented nerve cells than anode oriented nerve cells. In this way, waveform 200 can induce directional axonal growth in nerve cells.

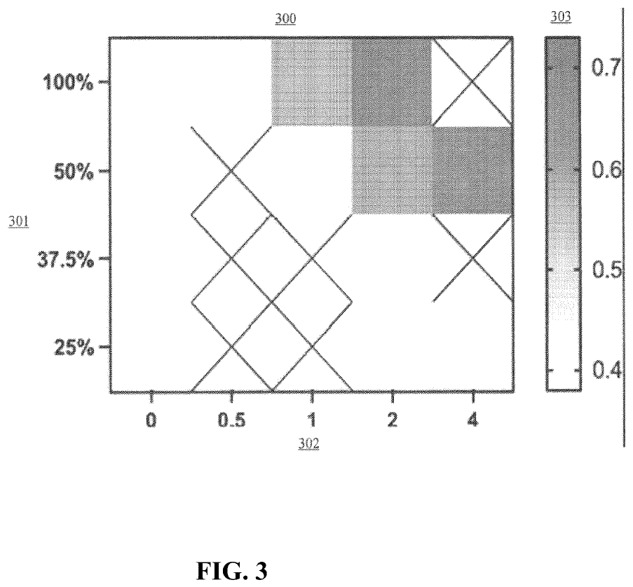

Turning ahead in the drawings, FIG. 3 displays an exemplary heatmap 300 summarizing data from exemplary monophasic pulses. In some embodiments, heatmap 300 can comprise y-axis 301, x-axis 302, and/or key 303. In further embodiments, heatmap 300 can define a parametric space for nerve growth inducing pulses in waveform 200 (FIG. 2). In this way, additional durations, structures, periods, amplitudes, widths, or other beneficial properties of waveform 200 (FIG. 2) can be obtained with little, if any, experimentation. In some embodiments, y-axis 301 can comprise a duty cycle. Generally speaking, a duty cycle can be understood as a ratio of a time a load or circuit is on compared to a time the load or circuit is off. In some embodiments, a circuit (e.g., a circuit created in system 100 (FIG. 1) can be considered off when the system implements a neutral polarity and/or a cathodic pulse. A circuit (e.g., a circuit created in system 100 (FIG. 1) can be considered off when the system implements an anodic pulse. In some embodiments, x-axis 302 can comprise a pulse amplitude in V/cm. Key 303 displays a value Q defining a ratio of nerve cells growing in a desired direction (e.g., towards optic chasm 103 (FIG. 3) as opposed to other directions. Cells in heatmap 300 with an X indicate parameters that were not tested.

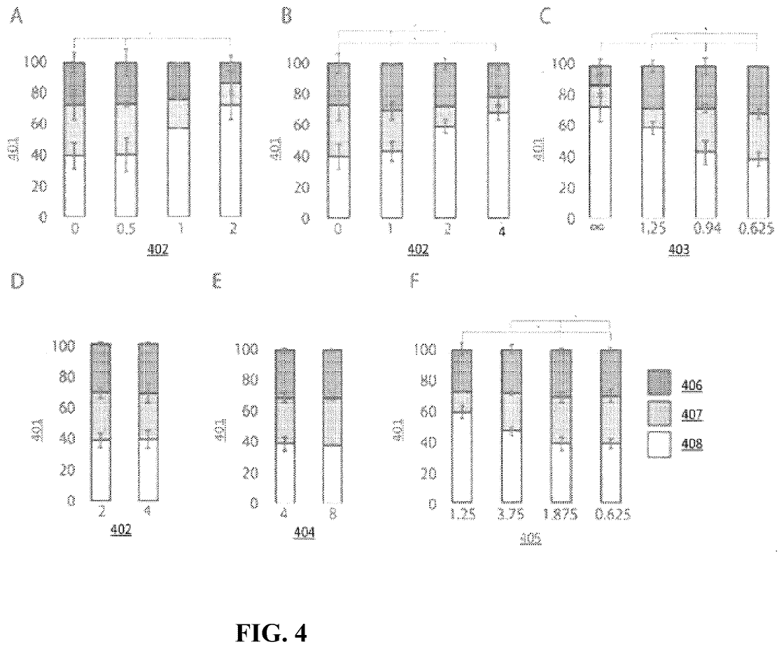

Turning now to FIG. 4, a graphical representation of nerve growth induced by different exemplary waveforms is shown. In some embodiments, axis 401 can comprise a percent axon growth, axis 402 can comprise a voltage gradient in V/cm, axis 403 can comprise a pulse width in seconds, axis 404 can comprise a current duration in hours, axis 405 can comprise interpulse delay in seconds, key 406 can comprise perpendicular nerve growth, key 407 can comprise anode directed nerve growth, and key 408 can comprise cathode directed nerve growth. As can be seen in FIG. 4A, DC experiments demonstrated that pulse amplitude plays an important role in determining whether a waveform will be effective at directing axon growth. As seen in FIG. 4B, purified RGCs directed their axons to grow towards a cathode when exposed to a monophasic waveform with a similar pulse width (2 V/cm, 400 mHz, 50% duty cycle). FIG. 4C, in turn, shows that decreases in pulse width below 1.25 secs were associated with proportional decreases in nerve growth. As can be seen in FIG. 4D, increasing an amplitude was unable to compensate for a loss of effect experienced from decreasing a pulse width. Even doubling an experimental timeline, as shown in FIG. 4E, or halving the interpulse interval (FIG. 4F, two right bars) was unable to compensate for a shorter pulse width. Conversely, increasing an interpulse interval for effective waveforms did not neutralize the effect of a waveform on directing RGC axon growth (FIG. 4F, two left bars). These experiments show that a pulse width threshold can be used to effectively direct axon growth. Pulse amplitude can also be used to determine a threshold effect on nerve growth. In some embodiments, doubling a stimulation amplitude from 2 V/cm to 4 V/cm did not result in a doubling of percent axons that grew towards a cathode (FIG. 4B). Therefore, when above a certain threshold, increasing stimulation amplitude does not have an additive effect on directing axon growth.

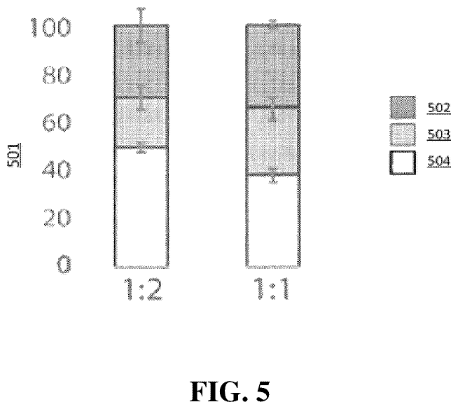

Turning ahead in the drawings, FIG. 5 illustrates a graphical representation of nerve growth induced by different exemplary waveforms. In some embodiments, axis 501 can comprise a percent axon growth, key 502 can comprise perpendicular nerve growth, key 503 can comprise anode directed nerve growth, and key 504 can comprise cathode directed nerve growth. In some embodiments, FIG. 5 can show that pairing monophasic waveforms that are effective at directing axon growth with monophasic waveforms that are ineffective, but of the opposite polarity, can cause RGC axon growth. Therefore, FIG. 5 displays results from an embodiment where a 2 V/cm, 400 mHz, 50% DC anodic working pulse with a −4 V/cm, 400 mHz, 25% DC cathodic recharging pulse (i.e. a 1:2 ACB). FIG. 5 shows that this waveform can be used to direct cathodic growth of purified RGC cells over controls. When a pulse amplitude to pulse width ratio was set to 1:1 (e.g., as in a traditional AC current), axonal galvanotaxis did not occur.

In various embodiments, the system includes a voltage source that has an active circuit. In some embodiments, an active circuit may increase a voltage between a ground electrode and a stimulation electrode while limiting a current between the ground electrode and stimulation electrode. In some embodiments, an active circuit may be a negative-equivalent resistance circuit. In various embodiments, a voltage source may have a capacitive circuit to increase a voltage between the ground electrode and stimulation electrode while limiting a current between the ground electrode and stimulation electrode. In some embodiments, a voltage and/or current source can have negative resistance. For example, a voltage or current source can comprise a non-Foster circuit. Generally speaking, a non-Foster circuit can be described as an active circuit with an equivalent negative resistance when attached in series to a passive system. In this way, an interface impedance between an electrode (e.g., stimulation electrode 102 FIG. 1) and tissue (e.g., optic nerve 104 (FIG. 1)) can be manipulated. In further embodiments, a non-Foster circuit can increase a voltage gradient along a nerve by increasing a capacitive conduction between a stimulation and ground electrode. Increasing capacitive conduction in a nerve circuit can then reduce a faradaic conduction of ions across an electrode-electrolyte interface, thereby preventing a reaction that causes electric field induced tissue damage. In some embodiments, a non-Foster circuit can be configured to increase a voltage difference between a stimulation electrode 102 (FIG. 1) and ground electrode 101 (FIG. 1) by 40% while delivering a same input current as a comparable Foster circuit.

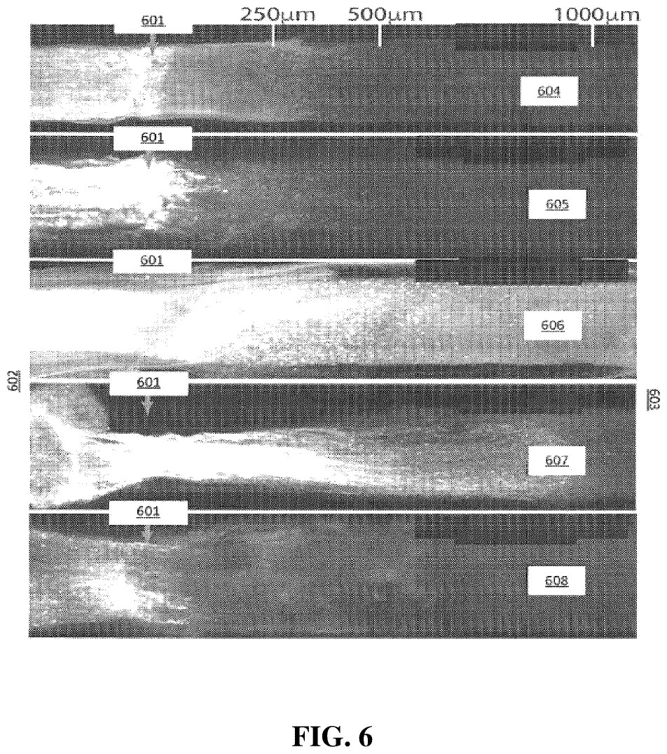

In some embodiments, waveform 200 (FIG. 2) implemented on system 100 (FIG. 1) can be used to treat nerve damage in a patient. Turning ahead in the drawings, FIG. 6 displays exemplary results of treatment using waveforms similar to waveform 200 (FIG. 2) implemented on systems similar to system 100 (FIG. 1) using parameters derived from heat map 300 (FIG. 3) in a rat model. In some embodiments, injury site 601 can comprise a location where a nerve was severed and/or damaged, lefthand side 602 can comprise a direction of an eye, righthand side 603 can comprise a direction towards a brain, nerve 604 can comprise an untreated nerve, and nerves 605-608 can comprise nerves treated with waveforms similar to waveform 200 (FIG. 2) using different parameters derived from heat map 300 (FIG. 3). In some embodiments, pulse widths ranging between 100 to 400 μseconds, amplitudes ranging between −400 to +400 μAmp, and/or a duty cycle of 50% can be used. As can be seen in FIG. 6, stimulation using waveforms similar to waveform 200 (FIG. 2) can cause nerve regeneration across a crush site. In some embodiments, treatment with waveforms similar to waveform 200 (FIG. 2) for 30 days can regenerate a nerve and restore eyesight. For example, 67% of rats whose optic nerves were crushed and then regenerated using electric field stimulation detected a visual cliff while no rats in an untreated group detected the visual cliff.

Exemplary embodiments of the present disclosure include apparatus and methods for non-invasive electrical stimulation for guided optic nerve regeneration. The optic nerve plays a crucial role in visual information processing by relaying signals from the retina to the brain. Diseases affecting the optic nerve, such as glaucoma, can severely impair vision due to the nerve's limited capacity for self-regeneration. One promising approach to promote nerve regeneration involves the use of electric fields to stimulate and guide axonal growth. The inventors' prior research demonstrated that electric field applied along a damaged optic nerve in a rat model promotes axons regeneration. While effective, this technique involves placing electrodes in direct contact with the optic nerve, posing challenges, such as requiring skilled surgeons and potential for tissue damage during implantation. Exemplary embodiments of the present disclosure therefore also include noninvasive methods for generating safe and effective electric fields leveraging computer simulations, validated with ex-vivo cadaveric measurements. As discussed herein, results show the promise of computational methods to correctly estimate the electric fields induced along the optic nerve by minimally invasive electroneural stimulators, thereby providing for the design of optimal systems that generate fields known to foster axonal growth.

### I. Introduction

The optic nerve (ON), comprising axons connecting the retina to the lateral geniculate nucleus (LGN) in the thalamus and other structures, plays a critical role in visual information processing. These axons are responsible for relaying signals from the retina's light-sensitive photoreceptors to the brain, enabling vision. Diseases affecting the optic nerve, such as glaucoma, can significantly impair vision. One of the primary challenges in addressing these conditions lies in the optic nerve's limited capacity for self-regeneration, spurring significant research on the development of potential therapeutic treatments to promote axonal regeneration.

In the pursuit of enhancing axonal regeneration within the central nervous system (CNS), substantial focus has been placed on mitigating the diminished regenerative capacity of adult neurons, attributed to a combination of cell intrinsic and cell extrinsic growth-impeding factors. Strategies have primarily centered on reactivating silenced developmental signaling pathways, particularly through targeting of mTOR pathway via PTEN inhibition or SOCS3 inhibition, to revert cells to a growth-permissive state [1]-[4]. Despite notable advances, accurately directing regenerating axons through the optic nerve continues to pose significant challenges, with frequent misrouting at critical junctions such as the optic chiasm. Other strategies more recently investigated, including the application of electric fields (EFs), show potential not only with promoting axonal growth but also in guiding axons to their appropriate targets, potentially enabling targeted regeneration within the CNS without the need for genetic modifications.

Many flavors of electrical stimulation have been explored in the literature as a potential treatment for vision recovery. Traditionally, brain computer interfaces have been designed to bypass damaged areas (e.g. Argus II to bypass damaged photoreceptors in retinitis pigmentosa) and relay visual information to intact downstream structures (retinal ganglion cells) [5]-[7]. Other devices, such as epiretinal, subretinal, and suprachoroidal, are also being explored for similar purposes [8]-[12]. Alternatively, Gall et al. presented clinical trial results on repetitive transorbital alternating current stimulation (rtACS) for partially sighted patients with optic nerve damage. Although patients demonstrated improvement in visual field performance, Gall et al. posited that their improvement was likely a result of reactivation of alpha frequency brain activity [13]. Few works, however, directly demonstrate employment of EF for axonal regeneration. A novel intraneural electrode array, OpticSELINE [14], was shown to be an effective stimulator of the optic nerve; rather than directing axon regeneration, however, it is thought to improve vision by bypassing traditional retinal pathways and directly activating the visual cortex.

Our research group has recently demonstrated that the application of specific electric fields along a damaged optic nerve in a rat model can promote directional nerve regeneration [15]. Although exciting in that results proved, for the first time, significant directional axonal growth in the optic nerve, in-vivo, and partial restoration of visual function, this technique is invasive as it involves implanting electrodes in direct contact with the nerve. Specifically, the approach utilizes a “J-shaped” (“hook”) stimulation electrode positioned at the base of the globe around the optic nerve, and a ground electrode implanted intracranially at the contralateral optic tract [16]. This methodology establishes a targeted EF along the ON, which is hypothesized to stimulate and promote axonal regrowth and repair [17]-[19]. Despite the potential efficacy of this method, it is noted that the requirement for direct nerve contact by the electrodes presents challenges, such as the invasiveness of the procedure and the potential risk of further nerve or tissue damage. This underscores the importance of continued research into noninvasive techniques that can be equally effective at promoting optic nerve regeneration without these associated risks.

Several parameters can influence the EF induced in a region between two electrodes (stimulation and ground): these include the input current, the dielectrics and tissue between electrodes, the size of the electrodes, and the distance and specific location of the electrodes. In the context of electrical stimulation of biological tissue, there is no opportunity to influence the conductivity between electrodes, which is determined by the tissue conductivity. Therefore, assuming a constant input current, only the physical characteristics and position of the electrodes are degrees of freedom that allow the inventors to influence the EF. Initiating human trials necessitates addressing safety and feasibility concerns, which this study aims to contribute to through computer simulations and ex-vivo measurements. This research explores different stimulation and ground electrodes to find the optimal configuration to generate EF along the optic nerve in humans, using either invasive or noninvasive methods.

In this study, the inventors developed and validated a computational model of electrical stimulation of the human head so that different electrode shapes and positions can be paired and their ability to generate an EF along the visual pathway predicted. This model will help fast-track translation of efforts to develop EF stimulation for neural restoration.

### II. Methods

### A. Cadaveric Conductivity Measurements

The human head is comprised of numerous tissue types, all with varied dielectric properties. When building computational models, such values are usually determined from existing databases (e.g. [20], [21]), which have the advantage of presenting frequency-dependent data in an easily accessible format, often sourced from one or more publications, in a single venue. Measurement of dielectric properties of tissues presents, however, unique challenges at low frequencies, with numerous works in the literature attempting to address such discrepancies [22].

As the inventors operate in the extreme low frequency [16], the simulations for all practical purposes can be considered quasi-static. In practice, this means that only the conductivity of the various tissues will impact the induced fields and current distributions in the human head model. Thus, conductivity measurements were conducted on cadaveric tissues using the 4-electrode method. This method is widely accepted for low-frequency tissue measurement; it also avoids the polarization of electrodes as the sensing and driving electrodes are distinct [23]. A probe was constructed, with 4 platinum electrodes 1 mm apart. This probe was used to measure the resistance of each tissue at 1 KHz using an LCR meter.

As the inventors' plan was to validate the inventors' model with direct measurements performed in cadavers, impedance measurements were performed with cadaveric tissue. Two measurements were conducted at different positions for each tissue. Two different sets of cadaveric tissue were measured, for a total of four measurements averaged together. The measured resistances were converted to conductivities as in and the resulting values are provided in Table 1 (shown below). For tissues that could not be measured, such as bone and blood vessels, conductivities provided by IT'IS' low frequency database were utilized. This database is separate from the database based on Gabriel's data [26], and includes tensor imaging data, as well as other newer sources.

### B. Electrode Configurations

Provided that the tissue between two electrodes is characterized by a uniform and isotropic conductivity, the largest induced EFs are found along a line connecting the two electrodes. This implies that, with implanted electrodes, the optimal location for the electrodes is in direct contact to the optic nerve. Additionally, the closer the electrodes are to each other, the higher the induced EF for equal stimulating current.

In considering possible effective locations for non-invasive stimulation of the optic nerve, an electrode placed directly on the cornea (for transcorneal stimulation), resembling a contact lens, is a convenient option [27], [28]. This location is advantageous as it is the closest point to the optic nerve without intervening bone, which is a poor conductor. Furthermore, an electrode on the cornea sits along the same axis as the optic nerve, having therefore the potential to generate a maximum EF along the nerve itself. The contact lens electrode considered for this purpose is a single turn ring of 6 mm outer radius and 5 mm inner radius.

Designing a noninvasive ground electrode presented instead the challenge in that there is no location on the human head where bone can be avoided in the path between the source and ground electrode. Although extracephalic ground montages show advantages compared to cephalic montages in the context of brain stimulation and this montage is used in vision restoration [13], based on the inventors' prior simulations, the most convenient location for the ground plate electrode for the application is on the scalp, at the back of the head. This location aligns with the optic nerve, facilitating the desired directional stimulation. The ground electrode considered here was modeled as a conductive square plate of size 16 cm2.

Besides these non-invasive electrode options, the inventors also considered electrodes that are in direct contact with the optic nerve. These electrodes were inspired from those used in [15] for experiments with rats but scaled to fit the human head. The source electrode was modeled as a 30 mm “J-shaped” stimulation electrode placed around the optic nerve at the base of the eye, while the ground as a 10 mm straight needle electrode placed in the contralateral optic tract.

Moreover, the inventors investigated the use of an endoendonasal electrode. This electrode consists of an L-shaped coated wire electrode, with metallization exposed for 10 mm at the end of the wire and placed along the ON near the orbital apex. The electrode is placed in between the J electrode and the intracranial electrode and can be used as either stimulation or ground. It is hypothesized that adding such electrode could strengthen the EF along the optic nerve.

### C. Quasistatic Computational Modeling

Given the low frequency of the treatment (1 KHz), quasi-static approximations are adequate for an accurate assessment of the current distribution within the tissue [30]. This approximation allows the inventors to forego the inclusion of inductive effects. Further, the inventors' primary interest in the current patterns within the tissue allows one to neglect capacitive effects, which are present at the electrode-tissue interface and are only essential to characterize transients—an aspect that is not a priority for the instant analysis. Thus, to optimize the efficiency of the computational method, the inventors have considered only the conductivity in each of the tissues, as in [31].

The quasi-static method that the inventors employed relies on a multiresolution version of the Admittance Method (AM), a computational method developed by the inventors' group [30], [32]-[34]. In its most general form, the multiscale AM relies on a varying voxelized grid of admittances which represent the resistivity and permittivity values of the tissues accounted for in the computational model. The currents and voltages in the resulting network, and therefore at all points in the computational model, are solved considering the specific source(s), ground(s), and stimulation current.

The Mida model (see FIG. 8), which is a heterogenous human head model with 0.5 mm resolution was the computational head model of choice because it provides a comprehensive representation of the human head, encompassing the eyes, ears, and deep brain structures, along with numerous distinct muscles, bones, skull layers, arteries, veins, cranial nerves, and salivary glands [35]. As cadaveric heads do not possess fluids such as cerebrospinal fluid (CSF), the inventors replaced CSF with neighboring white matter (WM) in the inventors' computational model of the human head.

### D. Electrical Input/Waveform

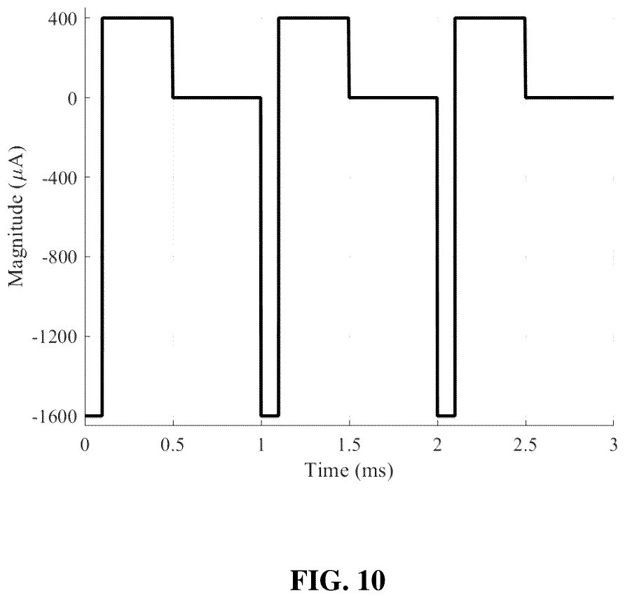

The stimulation method is current controlled with the input current of 400 μA. The anodic phase of the biphasic waveform shown in FIG. 10 was used as the input current in simulations. The rationale for this choice, detailed in the section II.F, is based on its dominant role in promoting axonal growth, as supported by findings in [1], [17], [19].

### E. Cadaveric Surgery

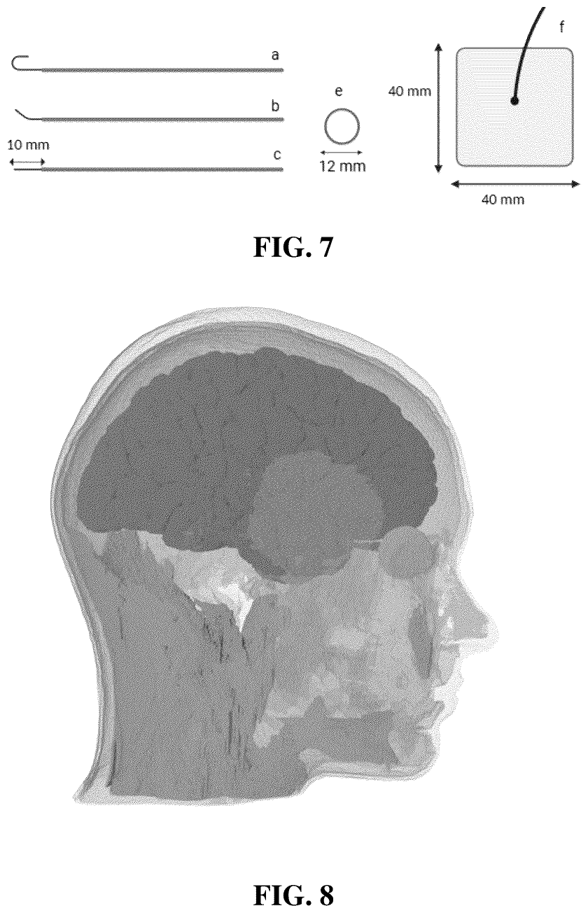

The inventors validated their computational model by performing voltage gradient measurements in cadaveric heads (University of California San Diego Body Donation Program). Electrodes used for stimulation are shown in FIG. 7 and fabricated according to specifications listed in Section II.B. FIG. 7 illustrates stimulation and ground electrodes used for EF guided optic nerve regeneration, specifically: (a) J stimulation electrode with 30 mm length; (b) L-shaped endonasal ground electrode with 10 mm length; (c) needle electrode ground with 10 mm length; (e) contact lens stimulation electrode with 6 mm radius; and (f) plate ground electrode with 40 mm length of each edge.

The contact lens electrode (ERG jet electrode from Diagnosys®) was placed on the corneal surface and secured into place with partial thickness scleral bites using 6-0 vicryl suture (Ethicon, Raritan, NJ). The J, needle, and endonasal electrodes were custom-made using tungsten wire with a 1 mm diameter, covered in heat shrink wrap, and selectively exposed as specified in Section II.B. The plate electrode is made from stainless steel.

The orbit was then accessed by performing a 360 circumferential limbal peritomy. The optic nerve was identified temporally by carefully reflecting the muscle cone and surrounding orbital fat. The J electrode was placed under the lateral rectus around the optic nerve; the distal end was secured to the orbit at the lateral canthus with 4-0 silk (Ethicon, Raritan, NJ).

To obtain access to the orbital apex, the inventors performed an extended endoscopic endonasal transsphenoidal approach, removing the lamina papyracea, the optic protuberance, sellar floor, and tuberculum sella to expose the optic nerve and chiasm. The endoendonasal electrode was placed endoscopically through the nasal cavity along the optic nerve and secured into place with Adherus dural sealant (Stryker, Kalamazoo, MI, USA).

To obtain access to the skull base, the inventors performed a standard pterional craniotomy to gain access to the optic nerve and chiasm. A needle-shaped intracranial electrode was placed inside the optic tract contralateral to the eye with the contact lens electrode.

A midline scalp incision was made, the scalp lifted off the skull with a freer elevator. The plate electrode, which was made by soldering a 100 mm copper wire (18 gauge, Striveday) to a 40 mm×40 mm stainless steel plate with 254 μm thickness (SAW 304, BPM) was positioned over the occipital bone. The wire electrode was threaded through a small incision made in the scalp. The scalp was then sutured together with 4-0 silk to prevent electrode movement.

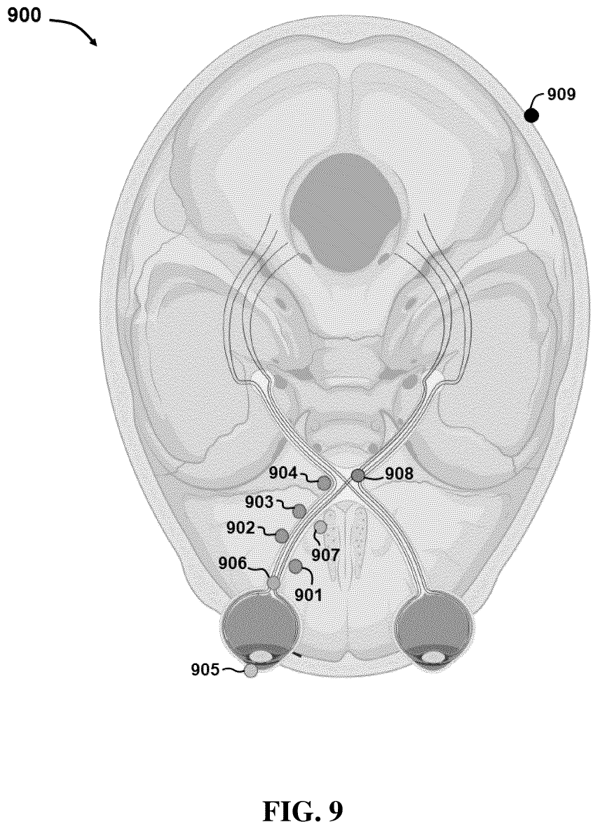

Measuring electrodes were placed along the anterior visual pathway. Specifically, a No. 11 scalpel blade was used to carefully puncture a small hold 1 mm behind the temporal limbus. A measuring electrode was inserted into the vitreous and secured into place with partial thickness scleral bites using 6-0 vicryl suture. Two measuring electrodes were placed in series along the optic nerve, one behind the globe which was secured while the second was placed deep in the orbit at the orbital apex. Measuring electrodes were placed along the ipsilateral and contralateral optic tracts. Another measuring electrode was placed along the rostral-caudal axis behind the optic chiasm. Another measuring electrode was placed in the thalamus. All electrodes were secured into place to the dura with 4-0 silk to prevent movement. A schematic of the electrode's location is shown in FIG. 9.

As shown in FIG. 9, one exemplary embodiment of a system 900 comprises measuring electrodes 901-904, a contact lens source electrode 905, and a J source electrode 906. In addition, system 900 comprises an endonasal ground electrode 907, a needle ground electrode 908 and a plate ground electrode 909. It is understood that the configuration shown in FIG. 9 is only exemplary and that other embodiments may comprise aspects different from those shown in FIG. 9. For example, the specific location and number of electrodes may be different from the embodiment shown. In addition, other embodiments may comprise electrodes with a different configuration from those shown and described herein.

### F. Voltage Measurements

For stimulation, the inventors utilized the MCS stimulus generator (STG 4008) in continuous current mode [36]. The input current is an asymmetric charge-balanced biphasic waveform with a frequency of 1 kHz, as depicted in FIG. 10. This current mirrors the one found to be effective during in-vivo experiments with rats [18]. In this case, however, the amplitude was increased fourfold for both anodic and cathodic phases. The rationale for selecting this amplitude in humans is based on the relationship between current amplitude used for deep brain stimulation in rats versus humans. As reported in [37], currents up to 400 μA were examined, with humans typically requiring 3-5 times this amplitude. Current was delivered via alligator clamps attached to each electrode.

Voltage measurements were performed utilizing deep brain stimulation (DBS) probes from Boston Scientific (2 DB-2202-45 with 4 contacts with 1 cm distance and 2 DB-2201-45DC with 8 contacts with 1 cm distance [38]. In DB-2201-45DC, 4 contacts are used interchangeably, making the distance between measurement points equal to 2 cm.

As shown in FIG. 9, two electrodes are located along the ON in the orbit, while two other electrodes are located along the ON after the orbit. Measurements were performed bilaterally on three cadaveric heads, totaling six eyes.

The inventors used a Keysight DSOX2014A oscilloscope for monitoring and recording the voltage, together with a custom switching circuit realized with one Arduino uno and 4 multiplexers (74HC4051) so as to be able to measure 16 points.

The inventors report the anodic component only as this is the phase that has been shown to be effective at driving axon growth [17]-[19].

### III. Results

### Cadaveric Conductivity Measurements

The inventors measured the conductivity of white matter, grey matter, muscle, fat, cornea, sclera, vitreous humor, lens and optic nerve as described in Section II.A. Table 1 compares the measured values with those provided by IT'IS in [25], which are based on Gabriel dispersion and IT'IS low frequency conductivity values. Coefficient of variation (CV), defined as the standard deviation divided by the arithmetic mean, was used to compare the measurements. The CV was used to account for the different scaling of the tissue properties. The tissues where the largest CV in conductivity was observed were white matter and fat.

### Computational Modeling of Induced Fields

TABLE 1
CONDUCTIVITY OF BIOLOGICAL TISSUES (S/M)

| | [symbol] data based on | [symbol] low frequency | Measured data | Number of | Coeff. of |
|---|---|---|---|---|---|
| Tissue | Gabriel disp [symbol] sion | conductivity data | from [symbol] | Samples | Variation |
| White Matter | 0.063 | 0.348 | 0.185 | 4 | 0.481 |
| Grey Matter | 0.099 | 0.419 | 0.225 | 4 | 0.357 |
| Muscle | 0.321 | 0.461 | 0.512 | 2 | 0.362 |
| Fat | 0.022 | 0.077 | 0.241 | 4 | 0.422 |
| Cornea | 0.423 | 0.620 | 0.722 | 2 | 0.367 |
| Sclera | 0.505 | 0.620 | 0.508 | 4 | 0.199 |
| Vitreous Humor | 1.500 | 2.160 | 1.614 | 4 | 0.283 |
| Lens | 0.200 | 0.345 | 0.382 | 4 | 0.250 |
| Optic Nerve | 0.029 | 0.348 | 0.569 | 4 | 0.039 |

[symbol]

indicates data missing or illegible when filed

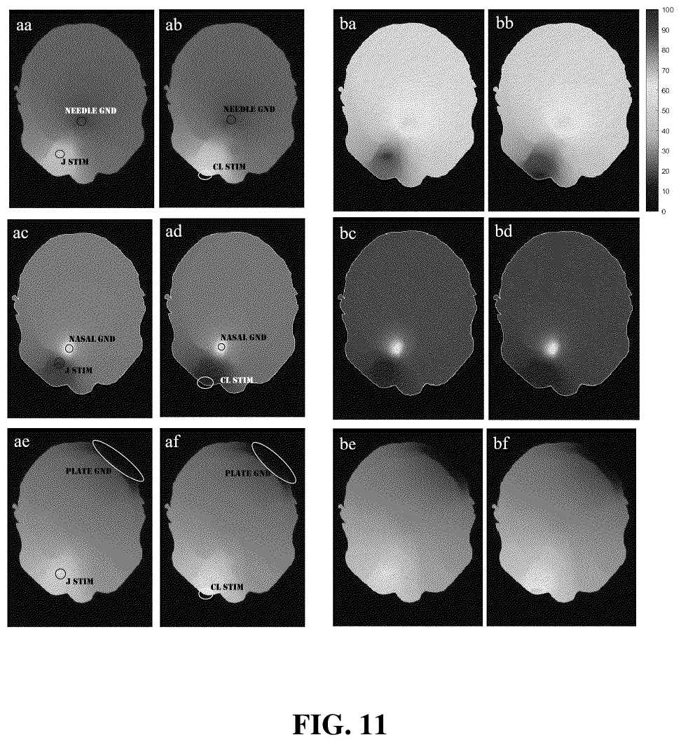

FIG. 11 shows the simulated voltage distribution at the orbitomeatal plane for 6 different electrode configurations, as obtained using the AM method described in Section II.C and D. Specifically, FIG. 11 illustrates computed voltage (mV) distribution at the orbitomeatal plane for: (aa) J stimulation-needle ground; (ab) Contact lens stimulation-needle ground; (ac) J stimulation-nasal ground; (ad) Contact lens stimulation-nasal ground; (ae) J stimulation-Plate ground; (af) Contact lens stimulation-Plate ground, performed including CSF to recapitulate the fields induced in the head of a potential patient. FIG. 11 also shows computed voltage (mV) distribution at the orbitomeatal plane performed with replacing CSF with white matter to recapitulate the fields induced in a cadaver head in: (ba) J stimulation—needle ground; (bb) Contact lens stimulation—needle ground: (bc) J stimulation—nasal ground; (bd) Contact lens stimulation—nasal ground; (be) J stimulation—Plate ground; (bf) Contact lens stimulation—Plate ground configurations.

In these experiments, simulations with (on the left side) and without CSF (on the right side) were performed as models with CSF would more closely resemble findings observed in a living patient while the model without CSF would most closely resemble the inventors' cadaveric experiments, against which the inventors planned to validate the inventors' model's findings.

The highest voltage is at the stimulation electrodes, and the lowest is near the ground electrodes, for both with and without CSF models. In fact, the voltage drops to zero for all ground electrodes. This, however, is not demonstrated displayed in FIG. 11 (ac), (ad), (bc) and (bd) as the endonasal ground is in a deeper plan than that which is displayed.

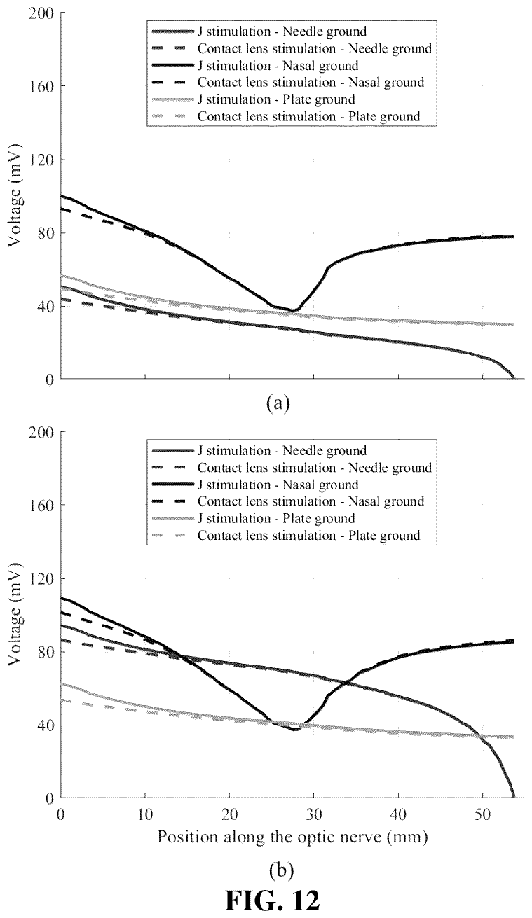

As described in the inventors' previous work [15], the potential for guided optic nerve regeneration is tied to the voltage distribution along the optic nerve. FIG. 12 shows such simulated voltage distribution for both cases in which the computational model includes CSF or does not include CSF. Simulations show that the electrode most impacted by the presence of CSF is the needle ground which terminates at the chiasm, an area surrounded by the CSF. FIG. 12 illustrates a graph showing simulated voltage along the ON of a human model for different combinations of direct and indirect stimulation and ground electrodes. FIG. 12 graph (a) illustrates voltage with CSF, while FIG. 12 graph (b) illustrates voltage without CSF. The stimulation is with a current source of amplitude 400 μA.

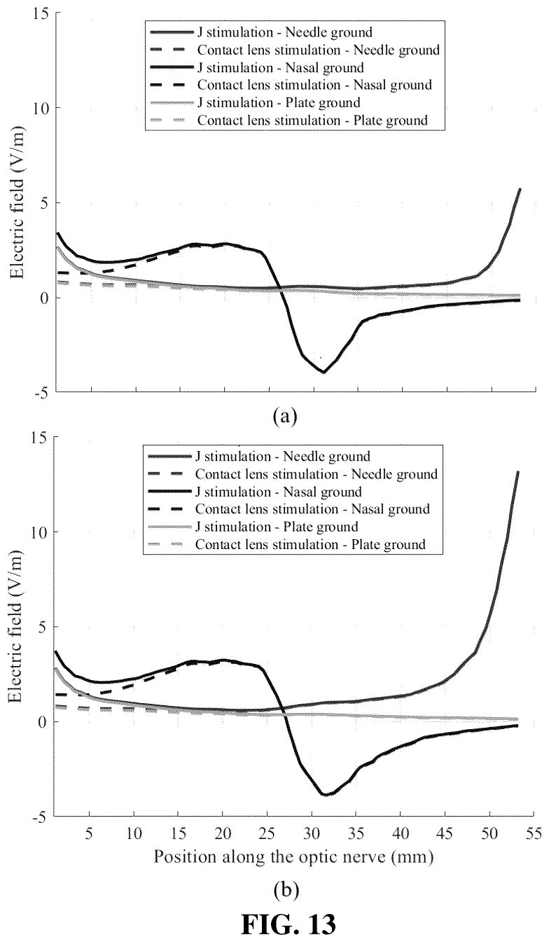

EF for the same models is instead plotted in FIG. 13. The cases with the needle electrode are once again the most impacted by the presence of CSF, particularly in the field intensity in the region proximal to the ground. Notably, absence of CSF increases the EF around the needle ground electrode by almost 100 percent. Specifically, FIG. 13 illustrates a graph showing simulated electric field along the ON of a human model for different combinations of direct and indirect stimulation and ground electrodes. In FIG. 13 graph (a) the simulated electric field is shown with CSF, while in FIG. 13 graph (b) the simulated electric field is shown without CSF. The stimulation is with a current source of amplitude 400 μA.

When using a CL electrode instead of the J electrode, the induced EF decreases. There are two important observations that can be made about this difference; first, in all setups, the largest decrease in EF occurs at the optic disc (where the optic nerve leaves the eye, i.e., x=0). In FIG. 13, this decrease continues until 20 mm away, after which the EF along the optic nerve is the same for both J and contact lens stimulation. Secondly, pairing the CL with the endonasal ground decreases the EF by 62\% compared to the J with endonasal. This difference between CL and J when paired with either needle or plate grounds is 71\%. This suggests that, as the ground gets farther from the stimulation electrode, the effect of the contact lens electrode becomes more significant but, eventually, this effect stabilizes. The same observations apply to the case without CSF.

When comparing the different grounds, the endonasal ground produces the largest EF along the optic nerve, followed by the needle and then the plate. This aligns with the principle that the EF between two electrodes decreases as the distance between them increases.

### Cadaveric Voltage Measurements

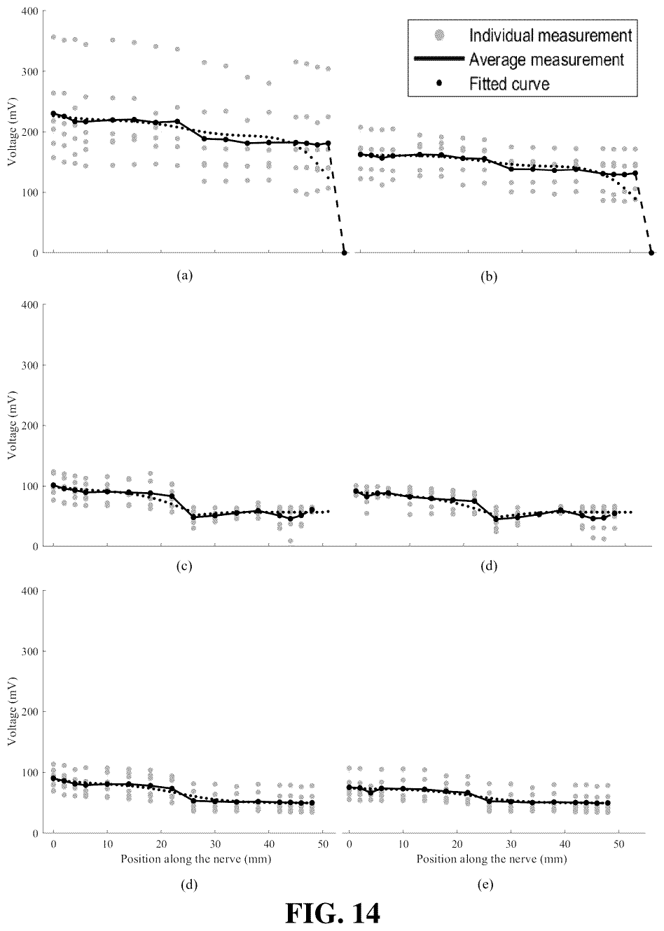

To validate the results provided by the simulations, measurements in cadaveric heads were performed as described in Section II.E and F. Measured voltages at several locations on the optic nerve are shown in FIG. 14. To mitigate the coarseness of the measured voltage, the inventors adopted a curve fitting procedure. In particular, FIG. 14 illustrates graphs showing measured voltage along the optic nerve of human cadavers, with a total of 6 measurements (Grey dots), the average (Black dots and line) and the fitted curve to the average measurement (small black dots). FIG. 14 (a) shows J stimulation-needle ground; FIG. 14 (b) shows contact lens stimulation-needle ground; FIG. 14 (c) shows J stimulation-nasal ground; FIG. 14 (d) shows contact lens stimulation-nasal ground; FIG. 14 (e) shows J stimulation-plate ground; and FIG. 14 (f) shows contact lens stimulation-plate ground.

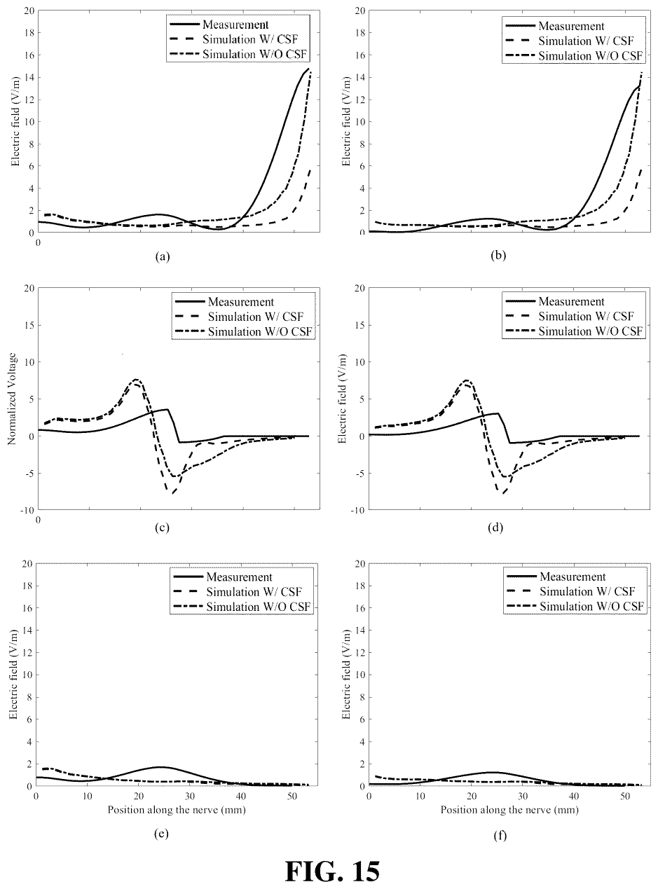

FIG. 15 compares the measured electric field (determined from the fitted curve of the measured voltage) to the simulated electric field, in the presence or absence of CSF, along the optic nerve. Results show a general consistency in values and profiles between measurement and simulation, particularly given the notable anatomical differences between the computational model of the human head and cadaveric heads. Specifically, FIG. 15 illustrates graphs showing measured electric field along the optic nerve (curve-fitted, from experimental measurements) compared against simulated electric field in a heterogeneous human head model, with and without CSF. The graphs correspond to the following configuration (stimulation electrode-ground electrode): FIG. 15 (a) J-needle; FIG. 15 (b) Contact lens-needle; FIG. 15 (c) J-nasal; FIG. 15 (d) contact lens-nasal; FIG. 15 (e) J-plate; and FIG. 15 (f) Contact lens-plate

As noted earlier, the absence of CSF had the greatest impact on cases with the needle ground electrode, and indeed, those measurements align more closely with simulations performed without CSF. Consistent with the inventors' simulations, cadaveric measurements demonstrate that using a CL stimulation electrode reduces the EF, with the maximum reduction observed at the optic disc. Furthermore, the endonasal ground produces the highest EF, while the plate ground generates the lowest.

### IV. Discussion

Embodiments of the present disclosure include a wearable or minimally invasive system to aid in the regeneration of axons of the optic nerve. Embodiments of the present disclosure comprise electric fields applied to the optic nerve through asymmetric biphasic stimulation waveforms described in that are effective at promoting directional growth to axons damaged by injury. Electrode configurations evaluated to date were performed in rats and demonstrated to be effective at mediating partial anatomical and electrophysiological restoration. This work employed a source electrode implanted behind the eye (J electrode) and a needle ground into the contralateral optic tract. If efficacy could be demonstrated with minimally invasive stimulation devices, such as with a contact lens and surface electrode, even if less efficacious than the implanted J and intracranial electrode combination, this could have a profound impact on the field of neuro-restoration as these surface electrodes are likely to carry a better safety profile.

Translating rodent findings to the human will invariably require more work than just scaling electrode size. Thus, the inventors developed and validated this computational model of electrical stimulation of the human head, both with CSF and without CSF, to be used as a tool by the community to streamline device design. The inventors' findings show that simulations closely resemble cadaveric measurements, with significant differences arising only when electrodes are positioned within the cerebrospinal fluid (CSF).

Our experiments show that the contact lens stimulating electrode produces smaller fields at the level of the optic disc (the region on the retina where optic nerve fibers converge and exit the eye) compared to those of the J electrode, regardless of the chosen ground. The reason for this is that the EF decreases rapidly with all electrode configurations. In the case of the CL electrode, the EF is already low by the time it reaches the optic nerve head, whereas with the J electrode, the EF is just beginning to take effect.

Given that in diseases like glaucoma and ischemic optic neuropathies, damage occurs to RGC axons and the low field strength in the region where axons exit and are expected to grow, the inventors' work raises the question of whether a wearable stimulating electrode can be effective. However, since the CL electrode has a larger surface area than the J electrode, it can also likely deliver a higher safe current based on the Shannon criteria [39]. Thus, it may be possible to compensate for the reduced EF by increasing the input current. This hypothesis is currently under active investigation by the inventors' group.

Another important factor in the electrical stimulation of the optic nerve is that the position and shape of the ground electrode significantly affect the field distribution. For instance, the plate electrode consistently generates the lowest EF, regardless of the paired stimulation source. This outcome could be attributed to the distance between the stimulation and ground electrodes or the large size of the plate. However, since the endonasal and needle electrodes are the same size, yet the needle consistently produces a lower EF than the endonasal electrode, the most plausible explanation is that the distance between the electrodes, rather than their size, has the greatest influence on EF amplitude.

Configurations that are able to minimize the distance between the stimulating and ground electrodes are more likely to be more effective at directing axon growth. While the endonasal ground presents as an attractive solution to this problem, given ease of placement by a skilled neurosurgeon, it is important to note that the EF direction behind the endonasal electrode (towards the brain) is negative, which is undesirable for axon regeneration. To address this, the endonasal electrode would need to also be used as the stimulation electrode and paired with another electrode, such as a needle or plate, as the ground. In other words, electrodes would likely need to be activated in series to drive axon growth.

It should be noted that, for the measurements in cadaveric heads, electrodes could not be removed after being positioned. Thus, for example, the endonasal ground electrode was left in position but disconnected when not in use. The presence of the metallization, although disconnected, could have had a minor effect on the results, as evidence by the undulation of the measured electric field around the 20 mm point (location of the disconnected endonasal electrode) in the cases of needle and plate electrodes.

For the inventors' conductivity measurements, the highest variation was observed for fat and white matter. Since fat has a very low conductivity, the inventors hypothesize that any water present in or around the fat causes the conductivity to jump unpredictably, as the water provides a small channel for ions to flow. In higher conductivity tissues, the conductivity difference between the tissue and fluids present is not as drastic, leading to smaller measurement variations. This hypothesis is corroborated by the work of Jones et al. [40], in which it was found that a surface layer of liquid can drastically affect the measurement on low conductivity tissues. In addition, white matter had especially high variation due to its anisotropy, which was not accounted for in the measurement.

When modeling EF stimulation of the visual pathway, the inventors attempted to more closely mimic the inventors' experimental set-up by removing CSF from computational models. While this appeared to be impactful for intracranial electrodes, especially for electrodes placed in CSF filled structures, little effect was noted when surface electrodes were modeled. In other words, when designing wearable surface electrodes, CSF properties appear to a play minor role in estimating the EF that can be induced in the human head. Furthermore, cadaveric measurements are adequate for estimating induced EFs with surface electrodes.

In summary, the inventors' quasi-static simulations may be used to design electrode configurations used to establish target induced fields in the optic nerve. This work validates the approach of utilizing such simulations for future studies of clinically relevant configurations for optic nerve growth, including the pursuit of multi-electrode configuration or the employment of inversion algorithms to determine electrode configurations necessary to achieve a desired induced electric field.

In this study, the inventors explored implanted and minimally invasive approaches for guided optic nerve regeneration by leveraging computational modeling and experimental validation. The inventors' work demonstrates the feasibility of generating targeted electric fields conducive to axonal growth, with simulations verified through cadaveric measurements. The results highlight the critical role of electrode placement and design, with configurations such as the endonasal ground showing promise for achieving effective field distributions while minimizing invasiveness.

The computational model developed and validated in this research provides a robust tool for predicting electric field behavior and optimizing electrode configurations. The strong alignment between simulations and measurements, particularly under experimental conditions without cerebrospinal fluid (CSF), underscores the accuracy and reliability of the model. Additionally, the findings emphasize the influence of electrode geometry and positioning on electric field strength and direction, offering valuable insights for advancing therapeutic applications.

These findings lay the groundwork for future investigations into multi-electrode systems and adaptive stimulation paradigms to enhance field uniformity and efficacy. Moving forward, integrating these results into in vivo models will be critical for evaluating the biological outcomes and safety of the proposed methods. Further exploration of strategies to address field localization and variability across tissue types will also be essential for clinical translation.

Although systems and methods for electric field directed nerve regeneration have been described with reference to specific embodiments, it will be understood by those skilled in the art that various changes may be made without departing from the spirit or scope of the disclosure. Accordingly, the disclosure of embodiments is intended to be illustrative of the scope of the disclosure and is not intended to be limiting. It is intended that the scope of the disclosure shall be limited only to the extent required by the appended claims. For example, to one of ordinary skill in the art, it will be readily apparent that any element of FIGS. 1-6 may be modified, and that the foregoing discussion of certain of these embodiments does not necessarily represent a complete description of all possible embodiments.

All elements claimed in any particular claim are essential to the embodiment claimed in that particular claim Consequently, replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

All of the devices, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
[1] K. K. Park, K. Liu, Y. Hu, P. D. Smith, C. Wang, B. Cai, B. Xu, L. Connolly, I. Kramvis, M. Sahin, et al., “Promoting axon regeneration in the adult cns by modulation of the pten/mtor pathway,” Science, vol. 322, no. 5903, pp. 963-966, 2008.
[2] J. Zhang, D. Yang, H. Huang, Y. Sun, and Y. Hu, “Coordination of necessary and permissive signals by pten inhibition for cns axon regeneration,” Frontiers in neuroscience, vol. 12, p. 558, 2018.
[3] L. Zhang, Z. Li, L. Mao, and H. Wang, “Circular rna in acute central nervous system injuries: a new target for therapeutic intervention,” Frontiers in molecular neuroscience, vol. 15, p. 816182, 2022.
[4] F. Sun, K. K. Park, S. Belin, D. Wang, T. Lu, G. Chen, K. Zhang, C. Yeung, G. Feng, B. A. Yankner, et al., “Sustained axon regeneration induced by co-deletion of pten and socs3,” Nature, vol. 480, no. 7377, pp. 372-375, 2011.
[5] M. S. Humayun, J. D.Weiland, G. Y. Fujii, R. Greenberg, R. Williamson, J. Little, B. Mech, V. Cimmarusti, G. Van Boemel, G. Dagnelie, et al., “Visual perception in a blind subject with a chronic microelectronic retinal prosthesis,” Vision research, vol. 43, no. 24, pp. 2573-2581, 2003.
[6] M. Humayun, R. Propst, E. de Juan, K. McCormick, and D. Hickingbotham, “Bipolar surface electrical stimulation of the vertebrate retina,” Archives of Ophthalmology, vol. 112, no. 1, pp. 110-116, 1994.
[7] M. S. Humayun, J. D. Weiland, G. Y. Fujii, R. Greenberg, R. Williamson, J. Little, B. Mech, V. Cimmarusti, G. Van Boemel, G. Dagnelie, et al., “Visual perception in a blind subject with a chronic microelectronic retinal prosthesis,” Vision research, vol. 43, no. 24, pp. 2573-2581, 2003.
[8] K. Stingl, R. Schippert, K. U. Bartz-Schmidt, D. Besch, C. L. Cottriall, T. L. Edwards, F. Gekeler, U. Greppmaier, K. Kiel, A. Koitschev, et al., “Interim results of a multicenter trial with the new electronic subretinal implant alpha ams in 15 patients blind from inherited retinal degenerations,” Frontiers in neuroscience, vol. 11, p. 445, 2017.
[9] R. Eckmiller, “Learning retina implants with epiretinal contacts,” Ophthalmic research, vol. 29, no. 5, pp. 281-289, 1997.

[10] K. Stingl, K. U. Bartz-Schmidt, D. Besch, C. K. Chee, C. L. Cottriall, F. Gekeler, M. Groppe, T. L. Jackson, R. E. MacLaren, A. Koitschev, et al., “Subretinal visual implant alpha ims-clinical trial interim report,” Vision research, vol. 111, pp. 149-160, 2015.

[11] E. Bloch, Y. Luo, and L. da Cruz, “Advances in retinal prosthesis systems,” Therapeutic advances in ophthalmology, vol. 11, p. 2515841418817501, 2019.

[12] T. Fujikado, “Retinal prosthesis by suprachoroidal-transretinal stimulation (sts), japanese approach,” Artificial vision: a clinical guide, pp. 139-150, 2017.

[13] C. Gall, S. Schmidt, M. P. Schittkowski, A. Antal, G. G. Ambrus, W. Paulus, M. Dannhauer, R. Michalik, A. Mante, M. Bola, et al., “Alternating current stimulation for vision restoration after optic nerve damage: a randomized clinical trial,” PloS one, vol. 11, no. 6, p. e0156134, 2016.

[14] V. Gaillet, A. Cutrone, P. Vagni, F. Artoni, S. A. R. Pinto, D. L. Di Paola, S. Micera, and D. Ghezzi, “Optic nerve intraneural stimulation allows selective visual cortex activation,” bioRxiv, p. 311035, 2018.

[15] M. Peng, P. Lam, M. Machnoor, J. Paknahad, E. Iseri, X. Shao, M. Shahidi, B. Thomas, G. Lazzi, and K. Gokoffski, “Electric fields direct full-length optic nerve regeneration and partial restoration of visual function,” Investigative Ophthalmology & Visual Science, vol. 63, no. 7, pp. 1139-1139, 2022.

[16] J. Paknahad, M. Machnoor, G. Lazzi, and K. K. Gokoffski, “The influence of electrode properties on induced voltage gradient along the rat optic nerve,” IEEE journal of electromagnetics, RF and microwaves in medicine and biology, vol. 6, no. 3, pp. 321-330, 2022.

[17] M. Peng, E. Iseri, A. Simonyan, P. Lam, T. Kim, S. Medvidovic, J. Paknahad, M. Machnoor, G. Lazzi, and K. Gokoffski, “Asymmetric charge balanced waveforms direct retinal ganglion cell axon growth,” Scientific Reports, vol. 13, no. 1, p. 13233, 2023.

[18] K. K. Gokoffski, X. Jia, D. Shvarts, G. Xia, and M. Zhao, “Physiologic electrical fields direct retinal ganglion cell axon growth in vitro,” Investigative ophthalmology & visual science, vol. 60, no. 10, pp. 3659-3668, 2019.

[19] T. Kim, E. Iseri, M. G. Peng, S. Medvidovic, T. Silliman, P. Pahla van, G. Niu, C. Huang, A. Simonyan, J. Pahnahad, P. Yao, P. Lam, V. Garimella, M. Shahidi, M. S. Bienkowski, D. J. Lee, B. Thomas, G. Lazzi, and K. K. Gokoffski, “Electric field stimulation directs targetspecific axon regeneration and partial restoration of vision after optic nerve crush injury,” Plos One, 2024.

[20] IT'IS Foundatio, “Dielectric properties.” https://itis.swiss/virtual-population/tissue-properties/database/dielectric-properties. Accessed: Oct. 25, 2024.

[21] IFAC, “Dielectric properties of body tissues.” http://niremf.ifac.cnr.it/tissprop/htmlclie/htmlclie.php. Accessed: Oct. 25, 2024.

[22] J. Zimmermann and U. van Rienen, “Ambiguity in the interpretation of the low-frequency dielectric properties of biological tissues,” Bioelectrochemistry, vol. 140, p. 107773, 2021.

[23] I. Kuznetcov, A. Kantzas, and S. Bryant, “Dielectric spectroscopy of nanofluids in deionized water: Method of removing electrode polarization effect,” Colloids and Surfaces A: Physicochemical and Engineering Aspects, vol. 647, p. 129039, 2022.

[24] I. Miccoli, F. Edler, H. Pfn'ur, and C. Tegenkamp, “The 100th anniversary of the four-point probe technique: the role of probe geometries in isotropic and anisotropic systems,” Journal of Physics: Condensed Matter, vol. 27, no. 22, p. 223201, 2015.

[25] IT'IS Foundation, “Low frequency conductivity.” https://itis.swiss/virtual-population/tissue-properties/database/low-frequency-conductivity/.

[26] C. Gabriel and S. Gabriel, “Compilation of the dielectric properties of body tissues at rf and microwave frequencies,” 1996.

[27] S. K. Agadagba, L. W. Lim, and L. L. H. Chan, “Advances in transcorneal electrical stimulation: from the eye to the brain,” Frontiers in Cellular Neuroscience, vol. 17, p. 1134857, 2023.

[28] S. K. Agadagba, A. B. Eldaly, and L. L. H. Chan, “Transcorneal electrical stimulation induces long-lasting enhancement of brain functional and directional connectivity in retinal degeneration mice,” Frontiers in Cellular Neuroscience, vol. 16, p. 785199, 2022.

[29] G. M. Noetscher, J. Yanamadala, S. N. Makarov, and A. Pascual-Leone, “Comparison of cephalic and extracephalic montages for transcranial direct current stimulation-a numerical study,” IEEE Transactions on Biomedical Engineering, vol. 61, no. 9, pp. 2488-2498, 2014.

[30] C. J. Cela, A multiresolution admittance method for large-scale bioelectromagnetic interactions. North Carolina State University, 2010.

[31] D. T. Brocker and W. M. Grill, “Principles of electrical stimulation of neural tissue,” Handbook of clinical neurology, vol. 116, pp. 3-18, 2013.

[32] A. Gonzalez Calle, J. Paknahad, D. Pollalis, P. Kosta, B. Thomas, B. Y. Tew, B. Salhia, S. Louie, G. Lazzi, and M. Humayun, “An extraocular electrical stimulation approach to slow down the progression of retinal degeneration in an animal model,” Scientific reports, vol. 13, no. 1, p. 15924, 2023.

[33] K. Loizos, “A multiscale computational modeling platform for design and analysis of electrical neural stimulation,” The University of Utah, 2017.

[34] C. Scott Bingham, “Simulating electrical stimulation and recording in a multiscale model of the hippocampus,” The University of Sothern California, 2019.

[35] IT′ IS Foundation, “Mida model.” https://itis.swiss/virtual-population/regional-human-models/mida-model/. Accessed: Aug. 8, 2024.

[36] Warner Instruments, “(stg4000 series) mcs stimulus generators.” https://www.warneronline.com/mcs-stimulus-generators. Accessed: Aug. 8, 2024.

[37] C. Hamani, M. Diwan, S. Isabella, A. M. Lozano, and J. N. Nobrega, “Effects of different stimulation parameters on the antidepressant-like response of medial prefrontal cortex deep brain stimulation in rats,” Journal of psychiatric research, vol. 44, no. 11, pp. 683-687, 2010.

[38] Boston Scientific, “Vercise™ deep brain stimulation systems product catalogue.” Catalog, Year. Accessed: Month Day, Year, if applicable.

[39] R. V. Shannon, “A model of safe levels for electrical stimulation,” IEEE Transactions on biomedical engineering, vol. 39, no. 4, pp. 424-426, 1992.

[40] D. Jones, R. Smallwood, D. Hose, and B. Brown, “Constraints on tetrapolar tissue impedance measurements,” Electronics Letters, vol. 37, no. 25, p. 1, 2001.

[41] Kim T., et al. (2025) Electric field stimulation directs target-specific axon regeneration and partial restoration of vision after optic nerve crush injury. PLOS ONE 20 (1): e0315562. https://doi.org/10.1371/journal.

1. A retinal ganglion cell (RGC) stimulation system for an optic nerve, the system comprising: a ground electrode located on a first side of optic nerve damage;
a stimulation electrode located on a second side of the optic nerve damage;
a voltage or current source connected to both the ground electrode and the stimulation electrode and configured to stimulate the stimulation electrode with an electrical waveform having a first voltage and a first current; and
a controller connected to the voltage or current source and controlling the first voltage and the first current of the electrical waveform.

2. The RGC stimulation system according to claim 1, wherein the first side of the optic nerve damage comprises an area behind an eye and the second side of the optic nerve damage comprises an area proximate to a frontal lobe of a brain or a temporal lobe of the brain.

3. The RGC stimulation system according to claim 1, wherein the first side of the optic nerve damage comprises an area in front of an eye and the second side of the optic nerve damage comprises an area proximate to an occipital lobe of a brain, wherein the ground electrode comprises a contact lens and the area in front of the eye comprises a cornea of the eye.

4. The RGC stimulation system according to claim 1, wherein the first side of the optic nerve damage comprises an area in front of an eye and the second side of the optic nerve damage comprises an area proximate to or within a nasal cavity, wherein the ground electrode comprises a contact lens and the area in front of the eye comprises a cornea of the eye.

5. (canceled)

6. The RGC stimulation system according to claim 1 further comprising a molecular scaffold located at the optic nerve damage.

7. (canceled)

8. The RGC stimulation system according to claim 1 further comprising a Ciliary neurotrophic factor (CTNF) containing implant placed proximate to the optic nerve damage.

9. (canceled)

10. (canceled)

11. The RGC stimulation system according to claim 1, wherein the electrical waveform is an asymmetric charge balanced biphasic waveform configured to promote neuronal regeneration of a retinal ganglion cell axon, wherein the first voltage changes over time.

12-16. (canceled)

17. The RGC stimulation system of claim 1, wherein the ground electrode and the stimulation electrode are selected from a group consisting of: (i) the ground electrode and the stimulation electrode are both platinum, (ii) the ground electrode and the stimulation electrode are both tungsten, and (iii) the ground electrode is tungsten and the stimulation electrode is platinum.

18. The RGC stimulation system of claim 1, wherein the electrical waveform comprises both positive pulses and negative pulses relative to a ground potential of the ground electrode, wherein the positive pulses have greater amplitude and shorter duration and the negative pulses have lower amplitude and longer duration.

19. The RGC stimulation system of claim 1, wherein the electrical waveform comprises both positive pulses and negative pulses relative to a ground potential of the ground electrode, wherein the negative pulses have greater amplitude and shorter duration and the positive pulses have lower amplitude and longer duration.

20. (canceled)

21. The RGC stimulation system of claim 1, wherein the electrical waveform comprises both positive pulses and negative pulses relative to a ground potential of the ground electrode, wherein the positive pulses and the negative pulses are of a same pulse length in time and a same pulse amplitude, wherein a combination of the positive pulses and the negative pulses promotes cellular health of a cell of the optic nerve.

22. The RGC stimulation system of claim 1, wherein the electrical waveform stimulates RGC axon growth toward an electrode of the ground electrode and the stimulation electrode has a positive voltage relative to the ground electrode.

23. The RGC stimulation system according to claim 1, wherein the electrical waveform is an asymmetric cathodic-first charge balanced biphasic waveform.

24. A method of retinal ganglion cell (RGC) stimulation for an optic nerve comprising: providing a ground electrode;
providing a stimulation electrode;
providing a voltage or current source connected to both the ground electrode and the stimulation electrode and configured to stimulate the stimulation electrode with an electrical waveform having a first voltage and a first current; and
controlling by a controller connected to the voltage or current source, the first voltage and the first current of the electrical waveform to generate a waveform, wherein the first voltage changes over time.

25. The method of RGC stimulation according to claim 24, wherein generating the waveform comprises:
generating both positive pulses and negative pulses relative to a ground potential of the ground electrode;
stimulating, by the positive pulses, neuronal regeneration of a retinal ganglion cell axon; and
restoring, by the negative pulses, a charge balance.

26. The method of RGC stimulation according to claim 25, wherein the negative pulses have greater amplitude and shorter duration and the positive pulses have lower amplitude and longer duration.

27. The method of RGC stimulation according to claim 24, where the waveform is an asymmetric charge balanced biphasic waveform to promote neuronal regeneration of a retinal ganglion cell axon in the optic nerve.

28. The method of RGC stimulation according to claim 24, where the waveform is a symmetric charge balanced biphasic waveform to promote cell health in the optic nerve.

29. The method of RGC stimulation according to claim 24, wherein providing the voltage or current source further comprises providing an active circuit and the method of RGC stimulation further comprises:
increasing, by the active circuit, the first voltage between the ground electrode and the stimulation electrode; and
limiting, by the active circuit, the first current between the ground electrode and the stimulation electrode.

30. A system for electric-field directed nerve stimulation comprising: a first electrode;
a second electrode;
a voltage or current source connected to both the first electrode and the second electrode and configured to stimulate the first electrode with an electrical waveform having a first voltage and a first current; and
a controller connected to the voltage or current source and controlling the first voltage and the first current of the electrical waveform to induce a voltage differential across a nerve for regeneration, wherein the electrical waveform comprises at least one of an asymmetric cathodic-first charge balanced biphasic waveform.

31. (canceled)
